(19) United States
(12) Patent Application Publication
(10) Pub. No.: US 2012/0149965 A1
(43) Pub. Date: Jun. 14, 2012

(54) Radiation Therapy Delivery System and Radiation Therapy Treatment Planning

(76) Inventors: Andrei Bronnikov, Westervoort (NL); Hans Meijs, Westervoort (NL)

(21) Appl. No.: 13/377,448

(22) PCT Filed: Jun. 11, 2010
(86) PCT No.: PCT/NL2010/050362

§ 371 Date: Feb. 24, 2012

(30) Foreign Application Priority Data
Jun. 11, 2009 (NL) 1037031

(51) Int. Cl. A61M 36/04

(52) U.S. Cl. 600/3

(57) ABSTRACT

The invention relates to a radiation therapy delivery system for use in effecting radiation therapy of a pre-selected anatomical portion of an animal body in which the delivery means displaces said at least one energy emitting source along a path through at least part of one of at least one catheter in a continuous motion using a pre-determined velocity profile along said paths, said velocity profile determined by solving an optimization problem in three-dimensions defined for a function describing the spatial dose distribution. The invention relates also to a radiation therapy treatment planning system and a method for generating a radiation treatment plan for use in effecting radiation therapy of an anatomical portion of an animal body in which the movement of the radiation source through the anatomical portion is continuous.

The invention relates to a radiation therapy delivery system for use in effecting radiation therapy of a pre-selected anatomical portion of an animal body.

The invention relates also to a radiation therapy treatment planning system as well as a method for generating a radiation treatment plan for use in effecting radiation therapy of an anatomical portion of an animal body.

The invention further relates to a method for generating a radiation treatment plan for use in effecting radiation therapy of an anatomical portion of an animal body.

The invention further relates to a computer program product for implementing the method as is set forth in the foregoing.

In brachy therapy treatment applications cancerous tissue, for example the male prostate gland, is canalized with one or more treatment catheters, often shaped as a hollow needle having a trocar tip. The treatment catheters are connected outside the patient's body with a so-called after loading apparatus having radiation delivery means for advancing one or more energy emitting sources through said catheters. The treatment planning solution generated by the treatment planning system prior to the treatment provides that the energy emitting source is stopped at pre-defined positions within the catheter, (and also generally inside the treatment site) for pre-defined times.

In general the pre-defined positions are known as dwell positions, and the pre-defined times at which the energy emitting source is halted at specific dwell positions are known as dwell times. Dwell positions and dwell times are calculated in a treatment planning unit by discrete optimization algorithms.

However treatment planning solutions containing amongst others a set of dwell positions and dwell times for the catheters to be inserted provide a discrete treatment solution. As each energy emitting source is stopped for a certain dwell time at each dwell position the radiation dose fraction in that position exhibits a point-like distribution of which the peak (or height) is determined by the length of the dwell time spent at said dwell position as well as other factors, such as the level of activity of the source.

Such a discrete treatment planning solution does not provide the ideal treatment planning solution, where it is intended that the target location receives a homogeneous dose coverage and where healthy tissue surrounding the target location is prevented from receiving radiation.

This invention relates to an approach in which the concept of the source stopping for finite dwell-times at discrete dwell-positions along the catheter's path is substantially replaced by a continuous movement along respective catheter's paths in a multi-catheter configuration. In particular, it is an object of the invention to provide accurate and reliable means for effectuating a desired dose distribution in three-dimensions using multiple catheters, wherein the shape of the desired dose distribution is more complicated than a sphere or a cylinder and having a substantial volume.

In accordance with an aspect of the invention radiation delivery means is provided for displacing said at least one energy emitting source along at least a portion of said respective paths through each of said catheters in a continuous motion using a pre-determined velocity profile determined for said paths, said velocity profile being calculated by solving an optimization problem in three-dimensions defined for a function describing the spatial dose distribution.

In a particular embodiment, the radiation dosing and treatment is realized by the system according to the invention adapted for moving the source through the catheter with a variable velocity, where the velocity varies with time (t).

An embodiment of a system for enabling irradiation of a substantially cylindrical small volume inside a vascular tree is known from EP 1 057 500. In the known system a sole catheter is used as the dose delivery depth is shallow, i.e. about 2 mm from the source along a 0.1 mm long portion of the vessel. The known system is accordingly adapted to move a radioactive source along a trajectory inside an intravascular catheter, the trajectory fully matching a longitudinal dimension of the lesion. Due to a simple geometry (a cylinder), the velocity for the source is determined based on a linear equation relating a dose rate of the source, a desired dose prescribed at 2 mm and a dimension of the lesion.

It will be appreciated that such linear approach is not appropriate for calculating three-dimensional dose distribution from a plurality of catheters, which may extend in a non-planar fashion with respect to each other, especially when the target volume has a complicated shape and is voluminous, for example has a volume of more than 1 cm³. It will be appreciated that for interstitial brachytherapy typical target volumes are about 5 cm³, some times even 10 cm³ or higher.

The technical nature of the invention is based on the following insights for enabling accurate computation of the velocity profiles along respective paths while conforming to the prescribed spatial dose distribution.

A source moving with varying velocity along the catheter can be envisaged as a source moving with constant speed but having a variable “intensity” along a path. This activity function along catheter I can be described as A_{I}(L_{I}). The total dose D(x) resulting from continuous moving sources through several catheters is

D(x)=Σ_{I}D_{I}(A_{I}(L_{I}),L_{I}),

where,
D(x) is the desired three-dimensional dose distribution function;
D_{I}(A_{I}(L_{I}), L_{I}) is the dose transfer function for the I^{th} catheter being a function of the shape of the Activity function A_{I}(L_{I}) along the catheter and catheter's path in 3D (L_{I}=L(x_{I})). A_{I}(L_{I}) is deduced from the source velocity v(t) along the catheter's path L_{I}(x), i.e. v(L).

According to a further insight, the three-dimensional dose distribution function D(x) may be resolved using a per se known Principle Component Analysis, in which any function B(x) can be described as the sum of weighted orthogonal functions bⱼ, i.e. B(x)=Σβⱼbⱼ(x). Generally, the harmonic functions bⱼ(x) are characterized by a restricted number of parameters. When using the appropriate orthogonal function set, the number of harmonics of the function Bⱼ(x) needed to describe B(x) will be restricted thus restricting the number of variables in the optimization equation, thus limiting the computational effort to find an optimal solution.

The activity along a catheter (A(L)), thus, can be decomposed in a series of principal components:

A(L)=ραᵢaᵢ(L).

When only the first 3 PCA components are used to describe the activity function A(L), A(L) is approached by A′(L) being:

A(L)≈A′(L)=α₁a₁(L)+α₂a₂(L)+α₃a₃(L).

Each function a₁(L), a₂(L) and a₃(L) is an activity function for which dose distributions can be computed, i.e. D₁(x), D₂(x) and D₃(x) respectively. It will be appreciated that according to adopted formalism the orthogonal functions may also be referred to as basis functions. The choice of the basis functions can be relatively arbitrary, providing that a sufficiently accurate representation is possible. As a good choice of the basis functions we can take a system of orthogonal polynomials such Chebyshev, Jacobi, Gegenbauer, Legendre, Laguerre, Hermit polynomials, but also elementary functions such as delta function, linear and piecewise step functions, or smoother functions such as sinc functions, Gaussian functions, spline and B-spline functions can be used. The functions may be or may not be strictly orthogonal. The choice of the function, however, will determine the accuracy of the representation and will influence the sufficient number of members of the representation. The latter may be important for the dimension of the optimization problem.

It is found to be advantageous to use lower order basis functions for resolving the spatial dose distribution function, as accurate computational results are achieved both in terms of desired three-dimensional dose distribution and the source velocity profiles along the defined paths for reasonable computation time. Accordingly, the previous equation for the dose distribution may be rewritten as:

D(x)≈D′^{N}(x)=α₁D₁(x)+α₂D₂(x)+ . . . +α_{N}D_{N}(x).

The number of harmonics needed to describe D(x) depends on the value of the residual ∥D(x)−D′^{N}(x)∥ or, as often done in numerical calculus, the difference between successive approximations: ∥D′^{N}(x)−D′^{N-1}(x)∥. It is found, that for obtaining good results it is sufficient to use the first 3 PCA components, i.e. three harmonics in the Fourier series.

It is further noted that the orthogonal PCA function used to develop a goal function for optimization algorithm can be chosen freely. When choosing a PCA function with a similar shape as the goal function, the lowest harmonic(s) will contain a large portion of the goal function and the contributions of the successive PCA harmonics will swiftly decrease. The better the fit between the goal function and the lowest harmonic, less harmonics are needed to describe the problem accurate enough. For physiological shapes (with relative low temporal frequencies), Chebyshev polynomials may be used and most time 5 to 7 harmonics suffice to describe more than 98% of the physiological shape.

In accordance to an aspect of the invention, the emitting source may be continuously displaced along all defined paths within the plurality of catheters provided in the target volume. Such displacement may be effectuated using pre-computed velocity profile, which may have either continuous or variable velocity. In addition, the velocity profile effectuated for at least a portion of a path may be combined with at least one dwell position.

Accordingly, the radiation therapy delivery system according to an aspect of the invention may further comprise a computing unit arranged for solving said optimization problem based on a related computing algorithm. Such computing algorithm may comprise PCA or any other suitable optimization method.

Hence by continuously displacing the energy emitting source through the catheters according to a pre-defined velocity profile a stepping function of a stepper motor may not be required as the energy emitting source is not stopped at the different dwell positions but advanced in a continuous or substantially continuous motion. Also the absence of any pre-planned dwell position (and associated dwell time) provides a continuous radiation dose distribution, which more accurately matches the ideal radiation dose distribution profile desired.

Preferably, the computing algorithm comprises a criterion for limiting allowable values of the velocity profile. Such a feature is advantageous as it limits the solution regarding the allowable velocity values to a range of feasible solutions in view of possible hardware limitations. Accordingly, very slow and very high local velocities are avoided. In addition, the computing algorithm may be arranged to allow a combination between a continuous displacement of the source along the respective paths and at least one dwell position of the source defined within the said paths.

The parameters defining the optimal dose in the continuous approach will be the same as for the known step-wise approach. The tumor (target area) should receive a high, homogeneous dose whereas the surroundings and especially the critical organs should be spared. In clinical practice, the contour of the tumor tissue is used as the boundary of the target area and the ideal solution (I(x)) is that the tissue inside the contour receives a perfect homogeneous 100% dose and the tissue outside the contour receives no dose. In practice this is not feasible, so the optimal solution O(x) will approach the ideal solution where parameters like realized homogeneity in the target area, steepness of the dose gradient at the contours and dose received in the surrounding tissue and critical organs are modelled in the optimization problem by reward and/or penalty functions.

Another parameter in the optimization is the number of catheters used. To restrict patient trauma, a plan using a low number of catheters is preferred. However, with a low number of catheters the homogeneity of the optimal solution will be restricted. Therefore, the number of catheters should be one of the parameters in the optimization process. To facilitate the latter optimization, the shape of the catheters and the spatial catheter distribution should be taken into the optimization equation as well.

Since the different constraints or factors may have opposing effects on the optimal solution, it becomes apparent that there is not a single optimal solution but there is a set of optimal solutions from which a clinician can choose based on his/her clinical insights and experience. Various means of visualizing this can be devised.

In essence, the contour of the tumor is the most important parameter in the optimization process: when dose is only delivered inside the tumor and not outside the tumor, the other area related constraints are met automatically. The second most important parameter is the homogeneity of the dose in the target volume and the third most important optimization parameter is the number of catheters used to realize the dose distribution.

Since the catheter parameters (number, shape and the distribution in the target area) define the shape and homogeneity of the dose, the optimization of the catheters in a wider sense as until now might be relevant when optimizing the dose pattern. The continuous approach will facilitate this optimization and the physical realization of the determined optimal dose distribution.

Although the process has been described with reference to a continuous movement of a source through a catheter, it should be appreciated that in order to obtain an optimum dose distribution in some circumstances it is necessary or may be desirable to halt or stop the source at one or more points in the catheter for a short period of time.

An alternative approach can be found in the concept of “slowness” of the movement of the source through the catheter. A mathematical model for the dose distribution and set up the optimization problem can be derived in such a way that allows the optimal distribution of the slowness, (or its reciprocal speed), of the sources in n catheters in terms of minimizing the error between the prescribed and received dose.

Consider a system of n catheters each of which contains a moving radioactive source with constant activity a and in which the geometry of catheters is known in the 3D space (x,y,z).

Let sᵢ(l) be the slowness of the source inside the i-th catheter at the distance l from the starting position of the source. The slowness is the inversion of the velocity of the source: sᵢ(l)=1/vᵢ(l). Let Aᵢ(l)=a×sᵢ(l) be the distribution of the activity for the i-th catheter. For interpretation of Aᵢ(l), note that Aᵢ(l)Δl=aΔtᵢ, where Δtᵢ is the time interval required for the source to move at the distance Δl. Assume that for each catheter we have the coordinate transformation (x,y,z)=Tᵢ(l), so that the position of the source can be determined in the 3D space as the function of l.

The dose at a certain point given by 3D vector (X,Y,Z) can be computed as

dᵢ(X,Y,Z)=∫(1/rᵢ²(l,X,Y,Z))×Aᵢ(l)dl=a×∫(1/rᵢ²(l,X,Y,Z))×sᵢ(l)dl, (1)

where rᵢ(l,X,Y,Z)=∥Tᵢ(l)−(X,Y,Z)∥(∥.∥ is the Euclidian norm) is the distance between the points given by the 3D vectors Tᵢ(l) and (X,Y,Z):

Conducting a discretization of equation (1) by representing sᵢ(l) by the finite series:

sᵢ(l)=Σcᵢⱼ×ψⱼ(l), i=1, . . . n, j=1, . . . m (2)

where ψⱼ(l) are a priori known basis functions and cᵢⱼ are unknown coefficients. Note that functions ψⱼ(l) may have positive and negative values to ensure accurate representation of sᵢ(l).

The examples of using series representation in the form of (2) come from all areas of physics. The choice of the basis functions can be relatively arbitrary, providing that a sufficiently accurate representation is possible. As a good choice of the basis functions a system of orthogonal polynomials such Chebyshev, Jacobi, Gegenbauer, Legendre, Laguerre, Hermit polynomials can be used. Also, elementary functions such as delta function, linear and piecewise step functions, or smoother functions such as sinc functions, Gaussian functions, spline and B-spline functions can be used. The functions may be or may not be strictly orthogonal. The choice of the function, however, will determine the accuracy of the representation and will influence the sufficient number of members of the representation. The latter is an important issue for the dimension of the optimization problem.

Using the series representation of the slowness in equation (1) for the dose:

$$\begin{matrix}{d}_{i} ( X , Y , Z ) = a \times \int ( 1 / {r}_{i}^{2} ( l , X , Y , Z ) ) \times \sum {c}_{ij} \times {ψ}_{j} ( l ) d l \\ = \sum {c}_{ij} \times [ a \times \int ( 1 / {r}_{i}^{2} ( l , X , Y , Z ) ) \times {ψ}_{j} ( l ) d l ] \\ = \sum {c}_{ij} \times {q}_{ij} ( X , Y , Z ) ,\end{matrix} \qquad ( 3 )$$

where

qᵢⱼ(X,Y,Z)=a×∫(1/rᵢ²(l,X,Y,Z))×ψⱼ(l)dl (4)

is the j-th “elementary” dose delivered by the i-th catheter. The values of qᵢⱼ(X,Y,Z) can be pre-computed using either numerical integration methods or analytical formulas if it is possible.

The total dose delivered by the i-th catheter is the sum of m elementary doses taken with coefficients cᵢⱼ. Assume that (X,Y,Z) belongs to a discrete set Ω that defines the region of the prescribed dose D(X,Y,Z).

The brachytherapy optimization problem can be defined as: Find such coefficients cᵢⱼ that minimize the discrepancy between the prescribed and computed dose distributions on Ω. After obtaining cᵢⱼ, compute the slowness via (2) and determine the velocity of the source as the function of l for each catheter.

Mathematically, the optimization problem can be defined as follows:

$${min}_{{c}_{ij}} ‖ D ( X , Y , Z ) - \sum \sum {c}_{ij} \times {q}_{ij} ( X , Y , Z ) ‖ , \\ ( X , Y , Z ) ∈ Ω , \\ i = 1 , … n , \\ j = 1 , … m , \qquad ( 5 )$$

where ∥.∥ stands for a certain norm.

In the case of the squared Euclidian norm ∥.∥² we deal with the least squares minimization problem

$${min}_{{c}_{ij}} {‖ D ( X , Y , Z ) - \sum \sum {c}_{ij} \times {q}_{ij} ( X , Y , Z ) ‖}^{2} , \\ ( X , Y , Z ) ∈ Ω , \\ i = 1 , … n , \\ j = 1 , … m , \qquad ( 6 )$$

that has a minimum-norm linear solution

c=Q⁺D (7)

based on the pseudoinverse Q⁺ of the matrix Q of the elementary doses. The size of Q is dim(Ω)×(m×n), the size of vector c is m×n and the size of vector D is dim(Ω).

Note that solution (7) does not necessarily provide numerical stability and/or non-negativity of the slowness. To ensure the numerical stability, the Tikhonov regularization method can be used, which gives

$${min}_{c} \{ {‖ D - {Q}_{c} ‖}^{2} + α {‖ Sc ‖}^{2} \} , \qquad ( 8 )$$

where S is the matrix of the stabilizing functional and α is the regularization parameter. To provide non-negativity of the slowness, additional constraint has to be added:

Σcᵢⱼ×ψⱼ(l)>0, i=1, . . . n, j=1, . . . m. (9)

Note that in the general case, the optimization problem can be reformulated subject to linear inequality constraints for the dose distribution in the region of interest and in the critical organs:

$${min}_{{c}_{ij}} ‖ D ( X , Y , Z ) - \sum \sum {c}_{ij} \times {q}_{ij} ( X , Y , Z ) ‖ , \\ ( X , Y , Z ) ∈ Ω , i = 1 , … n , \\ j = 1 , … m , \qquad ( 10 )$$

subject to

ΣΣcᵢⱼ×qᵢⱼ(X,Y,Z)≦D_{c}(X,Y,Z), (X,Y,Z)εΩ_{c}, (11)

Dₘᵢₙ(X,Y,Z)<ΣΣcᵢⱼ×qᵢⱼ(X,Y,Z)≦Dₘₐₓ(X,Y,Z), (X,Y,Z)εΩᵣₒᵢ, (12)

Σcᵢⱼ×ψⱼ(l)>0, (13)

where D_{c} is the critical dose in the region Ω_{c}, and Dₘᵢₙ and Dₘₐₓ are the minimum and maximum dose in the region of interest Ωᵣₒᵢ.

The optimization problem (10-13) can be resolved by any suitable mathematical algorithm for constrained optimization.

There are many known energy emitting sources which are capable of delivering the radiation therapy, examples include Iridium-192, Cobalt-60. Alternatively there are other so called “low energy emitting sources”, such as Ytterbium-170 or Iodine-125. The energy emitting source is not confined to being a radioisotope, but could also be an X-ray source. Small X-ray sources are known and could be used in place of a radioisotope.

A radiation therapy treatment planning system for use in effecting radiation therapy of a pre-selected anatomical portion of an animal body using a plurality of catheters disposed in three-dimensions having respective paths for accommodating at least one emitting source according to an aspect of the invention comprises:

input means for receiving image data corresponding to the anatomical portion to be treated;

treatment planning means for generating a radiation treatment plan for effecting said radiation therapy, said treatment plan at least comprising information concerning: the number, position and direction of said catheters within said anatomical portion to be treated, and a desirable spatial radiation dose distribution, wherein, said treatment planning means generates a radiation treatment plan in which said at least one energy emitting source is displaced along at least a portion of said respective paths through said catheters with a substantially continuous movement using a velocity profile, said velocity profile being determined by solving an optimization problem in three-dimensions defined for a spatial dose distribution function

A method according to the invention for generating a radiation treatment plan for use in effecting radiation therapy of an anatomical portion of an animal body, whereby a plurality of catheters is inserted in a certain orientation in three dimensions into said anatomical portion, each catheter defining a path for at least one energy emitting source moveable along said path through said catheter using radiation delivery means, said treatment plan including information concerning:

a number and corresponding orientations of said catheters within the anatomical portion to be treated;

one or more velocity profiles for each of said one or more catheters for said at least one energy emitting source;

a spatial radiation dose distribution for each of said catheters pursuant to the energy emitting source being displaced along said path through at least part of said catheter according to said velocity profile, the method comprising the step of determining said velocity profile by solving an optimization problem in three-dimensions defined for a spatial dose distribution function.

A computer program product according to the invention comprises instructions for causing a processor to carry out the steps of the above method. It will be appreciated that the computer program product may also comprise a suitable graphic user interface for enabling input and/or visualization of image date, preferably provided with information about the catheters spatial orientation and the respective paths therewithin. The computer program product may be further arranged to control a dose delivery system in accordance with the determined velocity profiles. This may be implemented using a suitable data transfer interface or by exporting data towards a control unit of the stepping motor of the dose delivery system.

These and other aspects of the invention will be discussed in more detail with reference to figures, wherein like reference numerals refer to like elements. It will be appreciated that the figures are presented for illustration purposes only and may not be used for limiting the scope of the appended claims.

### BRIEF DESCRIPTION OF THE DRAWINGS

The invention will now be described in more detail with reference to the accompanying drawings, in which:

FIG. 1 is a radiation therapy delivery system according to the state of the art;

FIGS. 2a and 2b show a discrete radiation dose distribution based on dwell positions and dwell times of an energy emitting source;

FIGS. 2c and 2d show a discrete and a continuous radiation dose distribution;

FIG. 3a shows a radiation dose distribution as can be found in practice and based on dwell positions and dwell times of an energy emitting source;

FIG. 3b shows a radiation dose distribution that can be found in practice when an energy emitting source is in continuous or substantially continuous motion through the target region or tumour;

FIGS. 4a and 4b show a radiation dose distribution as a function of velocity of the energy emitting source through the catheter against position in the catheter;

FIG. 5 an example of a radiation therapy treatment according to the present invention;

FIG. 6 radiation dose distributions according to the present invention.

### DETAILED DESCRIPTION OF THE DRAWINGS

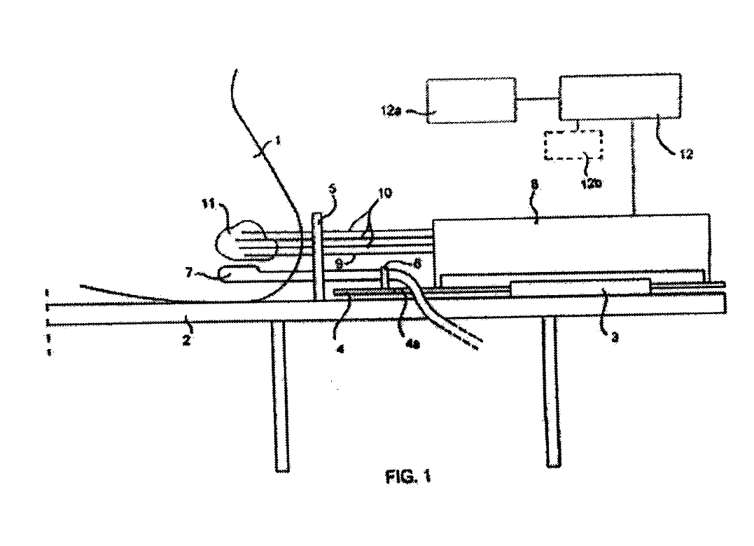

FIG. 1 shows in very schematic form various elements of a known radiation treatment delivery system for implanting an energy emitting source into a prostate gland. A patient 1 is shown lying in lithotomy position on a table 2. Fixedly connected to the table 2 is a housing 3. Housing 3 comprises a drive means 4 to move rod 4a stepwise. A template 5 is connected or mounted to the table 2, which template is provided (not shown) with a plurality of guiding holes through which holes hollow needles 9, 10 can be positioned relative to the patient. By means of a holder 6 a transrectal imaging probe 7 is fixedly connected to said rod 4a, which is moveable in a direction towards and from the patient by means of the drive means 4. The imaging probe 7 can be an ultrasound probe.

A needle 9 is used for fixing the prostate gland 11 in position relative to the template 5. A number of needles 10 are fixed into position through the template 5 in the prostate gland 11. The template 5 determines the relative positions of the needles 10 in two dimensions. The needles 10 are open at their distal ends and are sealed of by a plug of bio-compatible, preferably bio-absorbable wax. In said housing 3 a radiation delivery unit 8 is present.

A well-known therapy planning module 12a is provided for determining the desired number and orientation of said hollow needles as well as the relative positions of the energy emitting source(s) in each needle for displacement through said needle towards the prostate gland 11. Such therapy planning module 12a usually comprises a computer programmed with a therapy planning program. The therapy planning module 12a is connected to the radiation delivery unit 8 through a control device 12 for controlling the displacement of the one or more energy emitting sources through each needle. Control device 12 may be a separate device or may be an integrated part either of the radiation delivery unit 8 or of the therapy planning module 12a or may be embodied in the software of the therapy planning module 12a or of the radiation delivery unit 8.

The known device shown in FIG. 1 operates as follows. A patient 1 is under spinal or general anaesthesia and lies on the operating table 2 in lithotomy position. The (ultrasound) imaging probe 7 is introduced into the rectum and the probe is connected via signal line 7a with a well known image screen, where an image may be seen of the inside of the patient in particular of the prostate gland 11 as seen from the point of view of the imaging probe 7. The template 5 may be attached to the perineum of the patient to prevent or minimize any relative movement of the template and the prostate gland and the needles.

The drive means 4 is used to move the ultrasound probe longitudinally and also to rotate it to provide different angular images. The prostate gland 11 is fixed relative to the template 5 by means of one or more needles 9, 10. Subsequently further needles 10 are introduced in the body and the prostate gland under ultrasound guidance one by one.

Moving the imaging probe 7 with the drive means 4 longitudinally or rotationally within the rectum will provide the necessary images. After all needles 10 have been placed, their positions relative to the prostate gland 11 are determined in at least one of several known ways. In a known way the therapy planning module 12a uses information from the imaging probe 7 to confirm the actual position of the treatment needles 10 and then how the one or more energy emitting sources are to be displaced through each of the needles 10. The information from the planning module 12a about the displacement of the energy emitting sources through the needles 10 in terms of dwell positions and dwell times is used to control the radiation delivery unit 8.

In the known devices, energy emitting sources are displaced through catheter needles in a discrete manner, that is stepping motor means advance the energy emitting source in a stepwise manner between subsequent dwell positions, and the energy emitting source is maintained in each dwell position for a certain dwell time. The dwell time for each dwell position in general determines the amount of radiation delivered at each dwell position. Said radiation dose at subsequent dwell positions are to be considered as having a point-like distribution, the peaks of each radiation dose being dependent on the dwell time at said dwell position. The longer the dwell time, the higher the peak of the radiation dose at said dwell position.

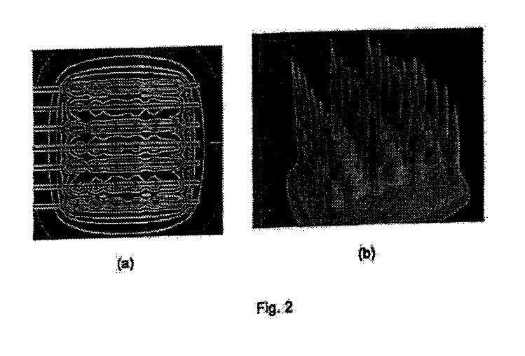

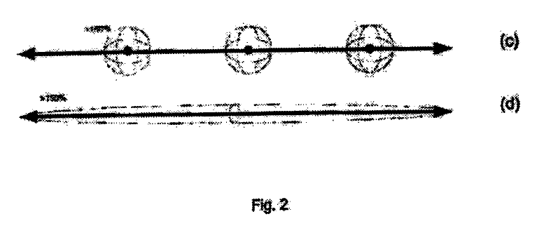

An example of a discrete radiation distribution profile resulting from the displacement of an energy emitting source through a catheter in a typical pattern of discrete dwell positions and dwell times is disclosed in FIGS. 2a and 2b. FIG. 2a shows a graphical depiction of an organ to be treated with several catheters implanted. Each catheter defines a path for an energy emitting source which is to be displaced in a discrete manner and to be stopped a specific dwell positions during pre-defined dwell times.

As clearly disclosed in FIG. 2b, an energy emitting source that stops at discrete dwell points along a path will generate peaks of radiation dose distribution or “hot-spots” around each dwell position. Although processing techniques can be used to smooth the hot spots in the radiation dose distribution a discrete planning solution as presently used provides a less accurate total dose coverage of the target volume (e.g. the male prostate gland or tumour in a female breast) to be treated.

When preparing one or more treatment planning solutions for a patient the contour of the target location (a tumor) is the most important parameter in the treatment planning optimization process. In order to avoid an unwanted, hazardous exposure to radiation of healthy (and sometimes fragile) tissue around the tumor it is preferred that radiation is only delivered inside the tumor and not outside the tumor.

Also the homogeneity of the radiation dose to be delivered to the target location is considered an important optimization parameter.

Another parameter in the planning optimization is the number of catheters used for the treatment. To limit trauma to the patient, a treatment plan using a low number of catheters is preferred. However, with a low number of catheters the homogeneity of the radiation distribution of the optimal solution will be restricted.

The parameters defining the optimal dose in the continuous approach will be the same as before: the tumor (target area) should receive a high, homogeneous dose whereas the surroundings and especially the critical organs should be spared. In clinical practice, the contour of the tumor tissue as detected by the use of imaging means is used to define and delineate the boundary of the target area. The ideal solution (l(x)) is that the tissue inside the boundary receives a perfect homogeneous 100% dose and the tissue outside the contour receives no dose.

In practice this is not feasible, so the optimal solution O(x) is intended to approach the ideal solution, where parameters such as realized homogeneity in the target area, steepness of the dose gradient at the boundary and dose received in the surrounding tissue and the critical organs are modeled in the optimization problem by reward and/or penalty functions.

Since the catheter parameters (number, shape and the distribution in the target area) define the shape and homogeneity of the dose, the optimization of the catheters in terms of the number and positioning will be relevant when optimizing the dose pattern.

According to the invention a treatment planning technique is proposed which provides a more accurate radiation dose distribution being conformal to the target location (volume to be treated) wherein the volume may have a complicated three-dimensional shape and be larger than 1 cm³. According to the treatment plan generated by the treatment planning means 12 said radiation delivery means 8 displaces said at least one energy emitting source along said path through at least part of one of said catheters 10 in a continuous motion and more in particular at a variable velocity wherein said velocity is computed based on an optimization algorithm as is discussed with reference to the foregoing. In particular, the method according to the invention is directed to generating a treatment plan, wherein a starting position and a finishing position along said path is defined, and that for each position between said starting position and said finishing position of said path a velocity profile for said at least one energy emitting source is defined.

Thus, instead of displacing an energy emitting source in a stepwise manner from dwell position to dwell position, with the method according to the invention the energy emitting source is displaced in a continuous manner through the catheter according to a velocity profile which more accurately matches the planned radiation dose distribution for said catheter. The continuous approach will facilitate this optimization and the physical realization of the determined optimal dose distribution.

For this continuous dose delivery approach a mathematical model for the dose distribution and setting up the optimization has been derived in such way, that the radiation treatment planning means can determine the optimal distribution of the slowness of the displacing source in the catheters in terms of minimizing the error between the prescribed and received dose.

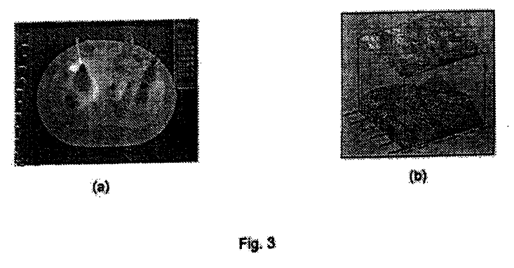

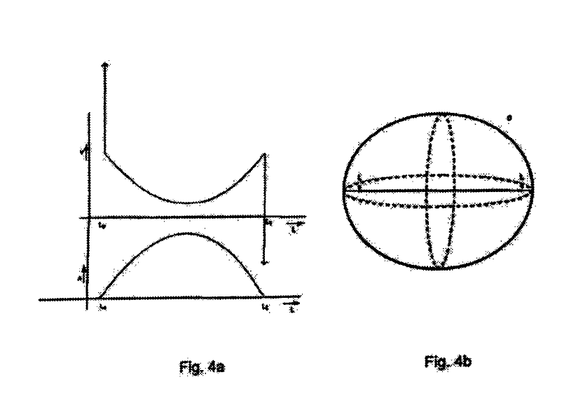

In the continuous dose delivery technique according to the invention, the radiation energy from the source will be spread along the path of the catheter 10 thus realizing a more homogeneous dose delivery in the target area 11 and less hot-spot volume in the target volume. So for this reason, the continuous dose delivery along the catheters' path is preferred over the discrete dose delivered in discrete dwell-positions. The difference between a discrete and a continuous dose distribution is shown in FIGS. 2c and 2d (as well as FIGS. 3a-3b).

FIG. 2c shows the radiation dose distribution resulting from a source delivering its dose in a typical pattern of discrete dwell-positions, whereas FIG. 2d discloses the radiation dose distribution resulting from a continuous moving source.

FIG. 3a shows a dose distribution as can be seen from a treatment plan in which there are a number of discrete dwell positions. It clearly depicts the hot-spots around the dwell positions, whereas FIG. 3b (and FIG. 2b) show a radiation distribution volume around the whole path of the continuously moving source. However, it will be appreciated that in some cases, wherein the computed optimal velocity profile comprises extremely low and extremely high values, it may be advantageous to combine the continuous volume and at least one dwell position within the defined paths.

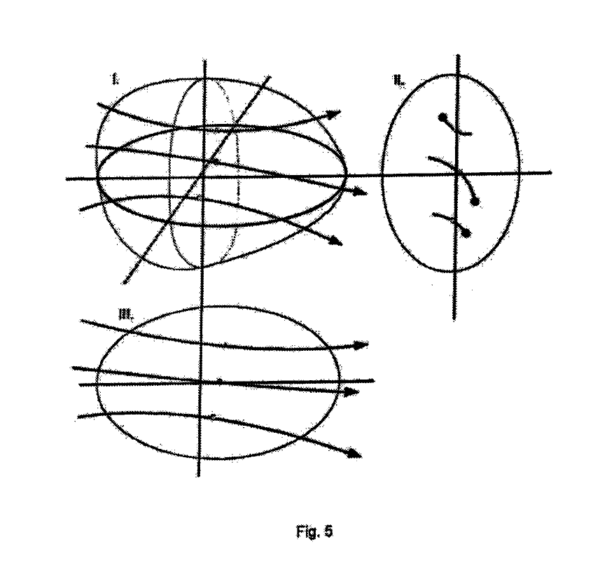

FIG. 4a shows at the top part a graph of the velocity of the source as a function of the position along the catheters' path v(L): The radioactive source is moved relatively fast to point L₀ from where its velocity is finite. The source slows down slowly till the middle of the catheter from where it accelerates till L₁. From L₁ it will be retracted to the safe. The second lower graph shows the activity A as a function of the position along the path of the catheter (A(L)), resulting from the source with velocity v(L).

FIG. 4b shows an Isodose surface of the dose distribution D(x) resulting from the activity A(L). In this FIG. 2, the 1 dimensional length along the catheter is used rather than the position of the catheter in 3D to simplify the figure. The catheter can have any shape in 3D.

FIG. 4a shows the velocity of a continuously displacing energy emitting source as a function of its position along a path L defined by the catheter through which the source is being displaced. The source is displaced by the radiation delivery means (unit 8 in FIG. 1) at a high speed to a starting position L₀ at the beginning of the treatment path L. At that starting position its velocity v will normally be large and the radiation being received by the target (depicted in FIG. 4 with activity A) is considered to be nil (or to be neglected). Starting from starting point L₀ the source is advanced at a decelerating speed v along the path toward a point halfway the catheter path at which point the velocity of the source is minimal. From that point halfway along the path the source is accelerated towards the finishing position L₁.

Upon arrival at the finishing position L₁ the source is retracted back into the radiation delivery unit 8 (FIG. 1) and stored in a radiation shielded compartment until the next treatment path through another catheter and following the same or another velocity profile according to the continuous treatment plan is determined and selected.

From FIG. 4 it will be appreciated that the radiation dose distribution A has a contour which is inversely proportional to the velocity profile v of the energy emitting source. A high velocity can be compared with a dwell time having a relatively short time interval, whereas a low velocity constitutes a dwell time of a relatively long time interval. Likewise a high velocity will result in a low radiation dose fraction at that position, whereas a slower moving source will be emitting a higher radiation dose fraction to its surrounding tissue.

According to the invention the treatment planning means 12 determines for each path (catheter 10 to be implanted in the tumor 11) a starting and finishing point for the continuous moving energy emitting source.

The parameters defining the optimal dose distribution for a treatment planning solution using a continuous moving energy emitting source are more or less the same as those for a known discrete treatment applications.

The tumor to be treated (target location 11) should receive a high, homogeneous radiation dose, whereas the surrounding, healthy (often fragile) tissue 1 and especially any nearby critical organ, such as a urethra, bladder, colon, or rectal sphincter should be spared. In clinical practice, the contour of the tumor tissue 11 is used as the boundary of the target area and the ideal solution is that the cancerous tissue inside the contour boundary receives a perfect homogeneous 100% radiation dose and the healthy tissue 1 outside the contour boundary receives no radiation dose.

In practice this is not feasible, so the optimal solution will approach the ideal solution, where parameters like realized homogeneity in the target area, the steepness of the radiation dose gradient at the contours (the defined starting and finishing points) and the radiation dose received in the surrounding tissue and the critical organs are modeled in the optimization process.

In FIG. 5 a target location (a tumor to be treated) is shown in 3 views and represented as an ovoid. In this example, 3 catheters are shown passing through it. When preparing a treatment planning solution for the treatment of the target, a small number of catheters is preferred, thereby reducing any trauma for the patient. However, with a small number of catheters the homogeneity of the optimal solution will be restricted, therefore optimization of the number of catheters being used is required. Also the shape of the catheters and the spatial distribution of the catheters with respect to each other and the target tumour (orientation relative to the target location) should be taken into account, when generating a continuous treatment planning solution.

Since the catheter parameters (number, shape and the distribution in the target area) define the shape and homogeneity of the dose, the optimization of the catheters is important when optimizing the dose pattern. The continuous movement approach will facilitate this optimization and the physical realization of the determined optimal dose distribution.

In FIG. 5 three imaginary catheters are planned in the target location. It is observed that the catheter paths in this example do not necessarily exhibit a straight line (as with a rigid hollow needle), but exhibit different curved orientations relative to the target location. However it is clear that beside straight, also curved catheters can be used or catheters exhibiting a helix or corkscrew shape.

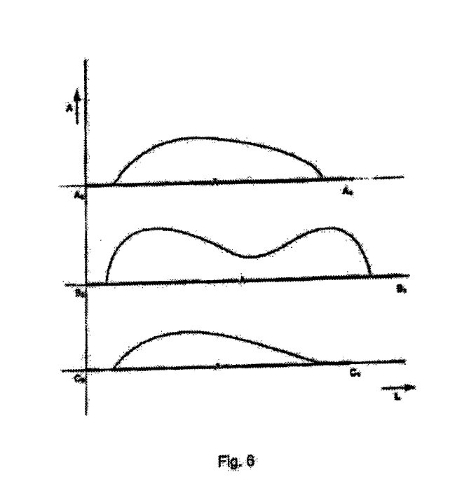

Using forward optimization algorithms the optimal radiation dose to be delivered by each catheter path is described, resulting in the radiation dose distributions A as shown in FIG. 6. Given the desired radiation dose distributions A the treatment planning means will define a starting and finishing point along the paths, and define a corresponding velocity profile for the energy emitting source to be displaced along said path, resulting in exposing the target location surrounding said path with the radiation dose distributions A as pre-planned. Normally, the energy emitting source will be moved through the catheter at a continuous, but varying speed, coming to a halt for a more or less longer period at each end of the defined path. It can be envisaged that there will be circumstances in which it is convenient for the energy emitting source to be brought to a halt or near halt at one or more points along a catheter path. Such a situation could arise if the optimization program determines it desirable in view of the number and positions of the catheters in relation to the size and shape of the tumour.

The invention has been described in relation to the treatment of cancer in a prostate gland. It is however equally valid and applicable for the treatment of any other body site suitable for treatment by brachytherapy, for example the treatment of breast cancer with women. In some treatments the imaging means will not be ultrasound imaging means, but could be X-ray or MRI imaging means.

Treatment delivery can be done by one of any known delivery or after loading apparatus but modified to be capable of operating in a substantially smooth continuous manner, driving or displacing the energy emitting source through the catheter between the defined starting and finishing points, if required with a variable velocity.

While specific embodiments have been described above, it will be appreciated that the invention may be practiced otherwise than as described. The descriptions above are intended to be illustrative, not limiting. Thus, it will be apparent to one skilled in the art that modifications may be made to the invention as described in the foregoing without departing from the scope of the claims set out below.

1. A radiation therapy delivery system for use in effecting radiation therapy of a pre-selected anatomical portion of an animal body for delivering a prescribed spatial dose distribution, said radiation therapy delivery system comprising:
a plurality of catheters conceived to be provided in said anatomical portion, each of said plurality of catheters defining at least one path for at least one energy emitting source; and
radiation delivery means for displacing said at least one energy emitting source along at least a portion of said respective paths through each of said catheters in a continuous motion using a pre-determined velocity profile along said paths, said velocity profile determined by solving an optimization problem in three-dimensions defined for a function describing the spatial dose distribution.

2. The radiation therapy delivery system according to claim 1, wherein the function describing the spatial dose distribution depends at least on the number of catheters, source activity function, source velocity, source path.

3. The radiation therapy delivery system according to claim 2, wherein the function describing the spatial dose distribution is resolved using Principle Component Analysis based on basis functions and related coefficients.

4. The radiation therapy delivery system according to claim 3, wherein a lower order basis functions are used for resolving the spatial dose distribution function, preferably, the lower three to seven basic functions

5. The radiation therapy delivery system according to claim 1, further comprising a computing unit arranged for solving said optimization problem based on a related computing algorithm.

6. The radiation therapy delivery system according to claim 5, wherein the related computing algorithm comprises a criterion for limiting allowable values of the velocity profile.

7. The radiation therapy delivery system according to claim 6, wherein the computing algorithm is arranged to allow a combination between a continuous displacement of the source and at least one dwell position of the source.

8. The radiation therapy delivery system according to claim 1, in which during said continuous motion said radiation delivery means displace said at least one energy emitting source along said path through at least part of one of said catheters at a variable velocity.

9. The radiation therapy delivery system according to claim 1, in which each path of said treatment delivery means has a defined starting position and a finishing position along said path and between said starting position and said finishing position along said path said at least one energy emitting source is moved with a defined velocity profile.

10. The radiation therapy delivery system according to claim 1, wherein the defined velocity profile for a specific catheter is inversely conformal to the radiation dose distribution of said catheter.

11. The radiation therapy delivery system according to claim 1, wherein said plurality of catheters are hollow needles.

12. A radiation therapy treatment planning system for use in effecting radiation therapy of a pre-selected anatomical portion of an animal body using a plurality of catheters disposed in three-dimensions having respective paths for accommodating at least one emitting source, said radiation therapy treatment planning system comprising:
input means for receiving image data corresponding to the anatomical portion to be treated; and
treatment planning means for generating a radiation treatment plan for effecting said radiation therapy, said treatment plan at least comprising information concerning: the number, position and direction of said catheters within said anatomical portion to be treated, and a desirable spatial radiation dose distribution, wherein, said treatment planning means generates a radiation treatment plan in which said at least one energy emitting source is displaced along at least a portion of said respective paths through said catheters with a substantially continuous movement using a velocity profile, said velocity profile being determined by solving an optimization problem in three-dimensions defined for a spatial dose distribution function.

13. The radiation therapy treatment planning system according to claim 12, wherein the spatial dose distribution function is resolved using Principle Component Analysis based on basis functions and related coefficients.

14. The radiation therapy treatment planning system according to claim 13, wherein a lower order basis functions are used for resolving the spatial dose distribution function, preferably, the lower three to seven basic functions.

15. The radiation therapy treatment planning system according to claim 12, further comprising a computing unit arranged for solving said optimization problem based on a related computing algorithm.

16. The radiation therapy treatment planning system according to claim 15, wherein the related computing algorithm comprises a criterion for limiting allowable values of the velocity profile.

17. The radiation therapy treatment planning system according to claim 16, wherein the computing algorithm is arranged to allow a combination between a continuous displacement of the source and at least one dwell position of the source.

18. The radiation therapy treatment planning system according to claim 12, and in which said treatment planning means generates a radiation treatment plan in which said at least one energy emitting source is displaced along said path through one of said catheters at a variable velocity.

19. A method for generating a radiation treatment plan for use in effecting radiation therapy of an anatomical portion of an animal body, whereby a plurality of catheters is inserted in a certain orientation in three dimensions into said anatomical portion, each catheter defining a path for at least one energy emitting source moveable along said path through said catheter using radiation delivery means, said treatment plan including information comprising:
a number and corresponding orientations of said catheters within the anatomical portion to be treated;
one or more velocity profiles for each of said one or more catheters for said at least one energy emitting source; and
a spatial radiation dose distribution for each of said catheters pursuant to the energy emitting source being displaced along said path through at least part of said catheter according to said velocity profile, wherein the method comprises the step of determining said velocity profile by solving an optimization problem in three-dimensions defined for a spatial dose distribution function.

20. A method according to claim 19, wherein the velocity profile of said at least one energy emitting source at a certain position along said path is inversely conformal to the radiation dose contribution of said energy emitting source in said position.

21. A method according to claim 19, wherein the treatment plan comprises a combination between a continuous displacement of the source and at least one dwell position of the source.

22. A computer program product comprising
instructions for causing a processor to carry out the steps of the method according to claim 19.

23. A computer program according to claim 22, arranged for outputting data or comprising instructions for controlling a radiation delivery means for displacing said at least one energy emitting source along at least a portion of said respective paths through each of said catheters in a continuous motion using a pre-determined velocity profile along said paths.
